(19) United States
(12) Patent Application Publication
(10) Pub. No.: US 2026/0369180 A1
(43) Pub. Date: Nov. 19, 2026

(54) METHOD FOR DEMONSTRATING THE MODE OF ACTION OF A SKIN CARE PRODUCT OR COMPONENT THEREOF

(71) Applicant: Conopco, Inc., d/b/a UNILEVER, Hoboken, NJ (US)

(72) Inventors: Suzanne Lynn GENCARELLI, Milford, CT (US); Bresny RECIO, Waterbury, CT (US); Cassandra Charlotte BRADFIELD, Neston (GB)

(73) Assignee: Conopco, Inc., d/b/a UNILEVER, Hoboken, NJ (US)

(21) Appl. No.: 19/158,732

(22) PCT Filed: Feb. 14, 2024
(86) PCT No.: PCT/EP2024/053759

§ 371 Date: Aug. 21, 2025

Related U.S. Application Data
(60) Provisional application No. 63/450,756, filed on Mar. 8, 2023.

(30) Foreign Application Priority Data
Apr. 17, 2023 (EP) 23168253.5

(51) Int. Cl. G09B 19/24

(52) U.S. Cl. CPC G09B 19/24

(57) ABSTRACT

A method demonstrating or assessing the mode of action of a skin care product or a component thereof, the method comprising: selecting a first substrate and a second substrate; combining the first substrate and second substrate in a container, wherein a surface layer predominately comprises the first substrate; applying a product to the surface layer, with the product in an effective amount, preferably to coat the surface layer; and visually assessing the second substrate, wherein the second substrate is capable of changing its appearance after interacting with the product.

### TECHNICAL FIELD OF THE INVENTION

The present invention relates to a method for demonstrating and/or assessing the mode of action of a skin care product or a component thereof.

### BACKGROUND OF THE INVENTION

Consumers have many choices when it comes to skin care products. To evaluate the efficacy of a skin care product, one characteristic that may be assessed is whether such product or the components thereof can penetrate through the topmost layer of skin (e.g., to/through the stratum corneum) which can yield some additional skin benefits compared to products that merely remain on the surface of the skin. Skin has three main layers, namely, the epidermis, the dermis, and the hypodermis, with the epidermis being the outermost layer. Within the epidermis, there are multiple layers, including the stratum corneum (which is the top layer of the epidermis) and the stratum lucidum, which is the next layer in the epidermis following the stratum corneum.

For example, the compositions disclosed in WO 2022/136357 (Unilever) penetrate into the stratum corneum and unexpectedly result in enhanced fatty acid elongation and/or lipid production deep in the stratum corneum and near the surface of the stratum lucidum. This surprisingly results in a long-lasting skin resilience barrier that is suitable to improve the overall health and appearance of skin. By contrast, topical products with complex lipids, such as ceramides, are large and not easily transported through the skin's outermost pores, and thus do not penetrate through the stratum corneum.

In vivo and in vitro instrumental measurement of, for example, fatty acid elongation and lipid production within the skin is often complicated and/or involve expensive laboratory equipment. Furthermore, such measurements often result in numerical parameters that are difficult for consumers to understand or at least relate to the efficacy of the product. Thus, there is a need to provide consumers with a simple and visually convincing demonstration of the mode of action of skin care products that does not require expensive or specialized equipment, particularly to be able to demonstrate/assess the ability of a skin care product or the components thereof to be able to penetrate through the epidermis and the benefits thereof.

### RELATED ART

D1: US2015/0132737 is directed to an in vitro or ex vivo method for preserving and/or keeping alive mammalian, preferably human skin biopsies, enabling to transport it and, if applicable, to culture it. Also disclosed is a skin biopsy thus preserved and obtained by such a method and relates to its use as a model especially in a kit for screening or selecting cosmetic or therapeutic compounds.

D2: son et al. is an article discussing an inquiry activity is to evaluate a variety of skin-care products.

### SUMMARY OF THE INVENTION

In one aspect, a method is provided for demonstrating and/or assessing the mode of action of a skin care product or a component thereof, the method comprising:
(i) selecting a first substrate and a second substrate;
(ii) combining the first substrate and second substrate in a container, wherein a surface layer predominately comprises the first substrate;
(iii) applying a product to the surface layer, with the product in an effective amount, preferably to coat the surface layer; and
(iv) visually assessing the second substrate,
   wherein the second substrate is capable of changing its appearance after interacting with the product.

In another aspect, a method is provided for comparing modes of actions for skin care products or components thereof, the method comprising:
(i) selecting a first substrate and a second substrate;
(ii) combining the first substrate and second substrate in at least a first container and a second container, wherein each container has a surface layer predominately comprising the first substrate;
(iii) applying a first product to the first container and a second product to the second container, preferably wherein the first and second products to coat the surface layer in each respective container, and preferably, wherein the first and second product is different in each container, and
(iv) assessing the second substrate in each of the first and second containers,
   wherein the second substrate is capable of changing its appearance after interacting with the products.

In yet another aspect, a method is provided displaying images and/or videos on visible display units for demonstrating, assessing and/or comparing modes of actions for skin care products or components thereof, the method comprises:
(i) selecting a first substrate and a second substrate;
(ii) combining the first substrate and second substrate in at least a first container and a second container, wherein each container has a surface layer predominately comprising the first substrate;
(iii) applying a first product to the first container and a second product to the second container, preferably wherein the first and second products to coat the surface layer in each respective container, and preferably, wherein the first and second product is different in each container;
(iv) assessing the second substrate in each of the first and second containers, wherein the second substrate is capable of changing its appearance after interacting with the products; and
(v) displaying images and/or videos of any previous step(s).

In yet another aspect, a method is provided for recording images and/or videos for demonstrating, assessing and/or comparing modes of actions for skin care products or components thereof, the method comprises:
(i) selecting a first substrate and a second substrate;
(ii) combining the first substrate and second substrate in at least a first container and a second container, wherein each container has a surface layer predominately comprising the first substrate;
(iii) applying a first product to the first container and a second product to the second container, preferably wherein the first and second products to coat the surface layer in each respective container, and preferably, wherein the first and second product is different in each container;
(iv) assessing the second substrate in each of the first and second containers, wherein the second substrate is capable of changing its appearance after interacting with the products; and
(v) recording the images and/or video of any of the previous step(s) on a recordable medium, and optionally displaying images and/or videos of any previous step(s).

All other aspects of the present invention will more readily become apparent upon considering the detailed description and examples which follow.

### BRIEF DESCRIPTION OF THE DRAWINGS

FIG. 1 shows a top view of two containers each with a first and second substrate, as described herein.

FIG. 2 shows a side view of two containers each with a first and second substrate, as described herein.

FIG. 3 shows the same two containers of FIG. 2 whereby one container has a first product (10a) the other container has a second product (10b).

FIG. 4 shows the same two containers with products 10a and 10b, respectively, of FIG. 3, after about 20 minutes.

### DETAILED DESCRIPTION

Except in the examples, or where otherwise explicitly indicated, all numbers in this description indicating amounts of material or conditions of reaction, physical properties of materials and/or use may optionally be understood as modified by the word “about”.

All amounts are by weight unless otherwise specified. It should be noted that in specifying any ranges of values, any particular upper value can be associated with any particular lower value.

For the avoidance of doubt, the word “comprising” is intended to mean “including” but not necessarily “consisting of” or “composed of”. In other words, the listed steps or options need not be exhaustive.

The disclosure of the invention as found herein is to be considered to cover all embodiments as found in the claims as being multiply dependent upon each other irrespective of the fact that claims may be found without multiple dependency or redundancy.

The methods described herein provide a visually compelling evaluation of the mode of action of skin care products or components thereof.

In one aspect, a method is provided for demonstrating or assessing the mode of action of a skin care product or a component thereof, the method comprising:
(i) selecting a first substrate and a second substrate;
(ii) combining the first substrate and second substrate in a container, wherein a surface layer predominately comprises the first substrate;
(iii) applying a product to the surface layer, with the product in an effective amount, preferably to coat the surface layer; and
(iv) visually assessing the second substrate,
   wherein the second substrate is capable of changing its appearance after interacting with the product.

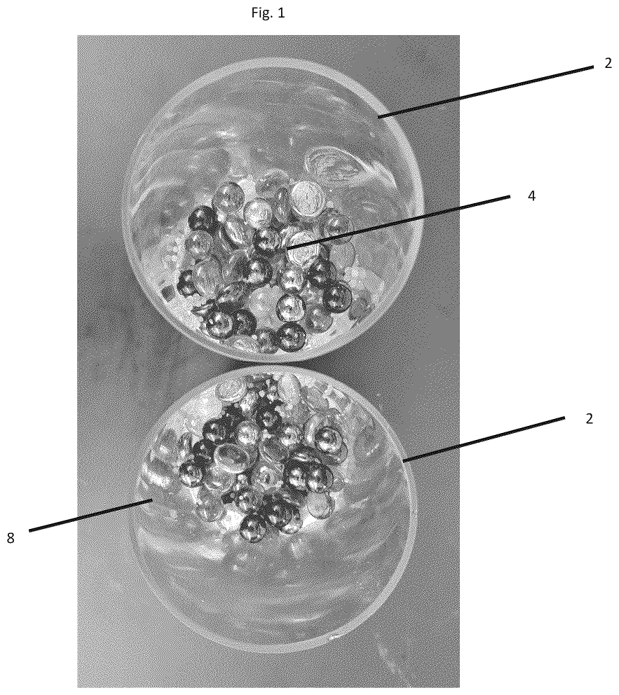

As used herein, “mode of action” refers to how the skin care product or component thereof achieves the effect in the human body. Moreover, the methods described herein allow for an easy comparison of different modes of action when done side-by-side, or when the results are recorded in photographs or video media. Preferably, the method is conducted by using a transparent container(s) (2), such as a glass or plastic, so it is possible to see the substrates used in the demonstration (see, e.g., FIGS. 1-4). When more than one container (2) is used, preferably the containers are the same size and shape. In one aspect, the diameter of the container (2) is between 1 inch and 40 inches, preferably from 1 inch to 20 inches, more preferably from 1 inch to 10 inches. In one aspect, the volume of the container (2) is 125 cubic inches to 40000 cubic inches, preferably 125 cubic inches to 20000 cubic inches, more preferably 125 cubic inches to 5000 cubic inches, even more preferably 125 cubic inches to 1000 cubic inches.

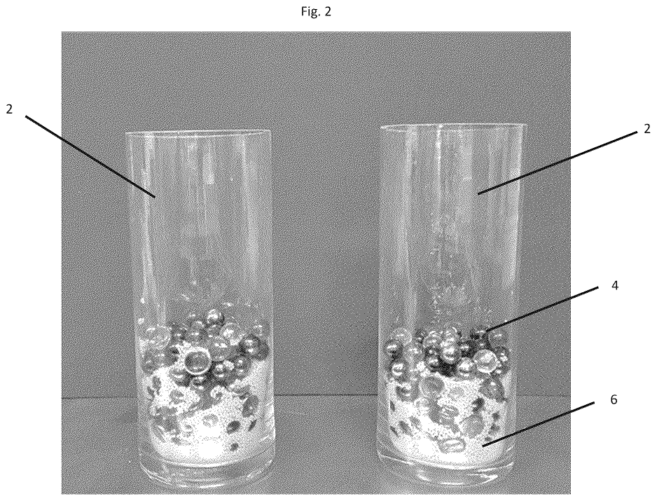

In one aspect, the method described herein has at least two different substrates. A first substrate (4) is a plurality of solid articles, preferably marbles, pebbles, stones, or any combination thereof. Preferably, the solid articles are non-absorbent. As used herein, “non-absorbent” refers to particles or articles that do not absorb liquid. Depending on the container(s) (2) used for the demonstration, the approximate diameter of the solid articles is 0.2 cm to 3 cm, or 0.5 cm to 2.5 cm, and any amount in between. For purposes of this demonstration, the first substrate (4) represents, for example, dead skin cells or the upper most layer of the epidermis. A second substrate (6) is one or more porous absorbent articles, preferably hydro beads (or water beads, such as Orbeez®) and/or other absorbent articles such as hydromorphic polymers, sponges, feminine hygiene products, diapers, or any combination thereof. Preferably, the second substrate (6) can not only absorb liquids, but will also swell and/or expand when liquid is absorbed. For purposes of this demonstration, the second substrate (6) represents, for example, the stratum corneum layer of the epidermis. In one aspect, the second substrate (6) is added to the bottom of the container, and then the first substrate (4) is added on top of the second substrate (6) (see, e.g., FIG. 2), such that there is a layer of predominately the first substrate (4) being closest to the opening of the container, e.g., a surface layer (8) (see, e.g., FIG. 1). As used herein, “predominately” means at least 85%, or at least 90%, or at least 95%, or at least 99% of the surface layer (8) is the first substrate (4). In one aspect, the weight ratio of the first substrate (4) to the second substrate (6) is about 6:1 to about 1.5:1, or about 4:1 to about 1.5:1, or about 3:1 to about 1.5:1, or about 2:1 to about 1.5:1. In one aspect, the first substrate (4) is larger in size than the second substrate (6). In one aspect, the first substrate (4) and second substrate (6) are different colors.

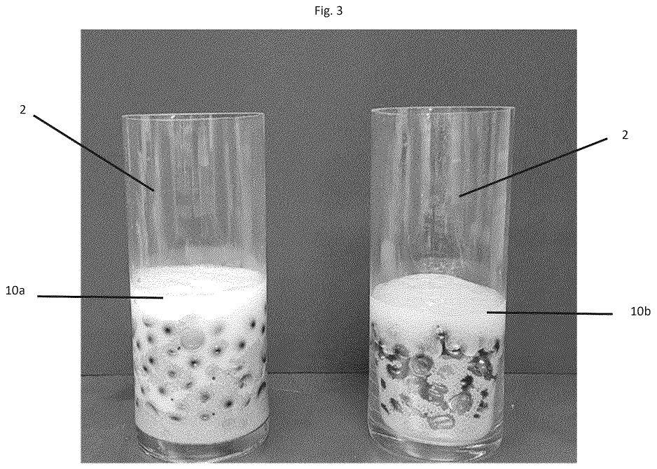

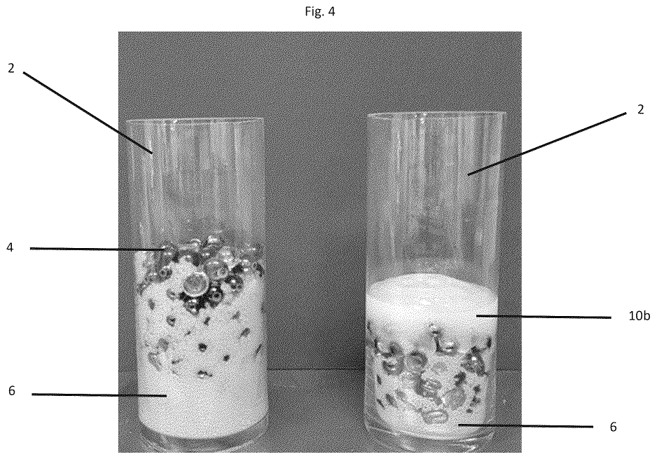

The method requires applying a product (10) to the surface layer (8) in the container. For purposes of this method, the demonstration utilizes a product intended to demonstrate the mode of action of a skin care product or component thereof. In one aspect, the product (10a) has a viscosity of 0 to 100 mPa·s, or 1 to 50 mPa·s, and is able to permeate through the first substrate (4) (see, e.g., FIG. 3) such that the second substrate (6) will absorb at least some of the product (10a), and preferably, the second substrate (6) will expand and/or swell, which can be visually assessed (see, e.g., FIG. 4). In one aspect, the product (10a) is water, opacified water, milk, juice or any combination thereof. In one aspect, it is possible to measure how much the second substrate (6) has swelled or expanded by measuring the increase in area or volume for the second substrate (6).

Alternatively, the product (10b) has a viscosity of 101 to 40000 mPa·s, or 5000 to 20000 mPa. s, and does not permeate through the first substrate (4) or has limited ability to permeate through the first substrate, which can also be visually assessed (see, e.g., FIGS. 3 and 4). As used herein, “a limited ability to permeate” means that the product (10b) generally remains on the surface layer (8) and there is no noticeable change in the second substrate (6) throughout the demonstration period. In one aspect, the product (10b) is water comprising 3-25%, preferably 3-10%, of a thickening agent, yogurt, apple sauce or any combination thereof. As used herein, a thickening agent may refer to polysaccharides, including starches, natural/synthetic gums, cellulosics, and any combinations thereof. Representative of the starches are chemically modified starches such as sodium hydroxypropyl starch phosphate and aluminium starch octenylsuccinate. Suitable gums include xanthan, sclerotium, pectin, karaya, Arabic, agar, guar, carrageenan, alginate and combinations thereof. Suitable cellulosics include hydroxypropyl cellulose, hydroxypropyl methylcellulose, ethylcellulose and sodium carboxy methylcellulose. Synthetic polymers are yet another class of effective thickening agent. This category includes crosslinked polyacrylates such as the Carbomers, polyacrylamides such as Sepigel® 305 and taurate copolymers such as Simulgel EG® and Aristoflex® AVC, the copolymers being identified by respective INCI nomenclature as Sodium Acrylate/Sodium Acryloldimethyl Taurate and Acryloyl Dimethyltaurate/Vinyl Pyrrolidone Copolymer. Another preferred synthetic polymer suitable for thickening is an acrylate-based polymer made commercially available by Seppic and sold under the name Simulgel IN100. Polyquaternium 32 and/or 37 are also suitable for use. Cellulose microfibrils may be used as thickening agents and these include secondary cell wall materials (e.g., wood pulp, cotton), bacterial cellulose, and primary cell wall materials. Often the source of primary cell wall material is selected from parenchymal tissue from fruits, roots, bulbs, tubers, seeds, leaves and combination thereof; more preferably is selected from citrus fruit, tomato fruit, peach fruit, pumpkin fruit, kiwi fruit, apple fruit, mango fruit, sugar beet, beet root, turnip, parsnip, maize, oat, wheat, peas and combinations thereof; and even more preferably is selected from citrus fruit, tomato fruit and combinations thereof. A most preferred source of primary cell wall material is parenchymal tissue from citrus fruit. Citrus fibers, such as those made available by Herbacel® as AQ Plus can also be used as source for cellulose microfibrils. The cellulose sources can be surface modified by any of the known methods including those described in Kalia et al., “Nanofibrillated cellulose: surface modification and potential applications”, Colloidal Polymer Science, Vol. 292, pp. 5-31 (2014).

Depending on the substrates and product(s) that are used, the demonstration period can be from 1 minute to 90 minutes, or 1 minute to 60 minutes, or 1 minute to 30 minutes.

The preference for waiting longer periods of time is so that visual aspect of the appearance change of the second substrate, if any, is more pronounced and more easily seen by the consumer.

In a preferred aspect, at least two containers are used, each with the same first and second substrate, yet different products are used. A visual assessment to compare each of the second substrates can occur.

In one aspect, the method comprises a step of capturing images and/or shooting videos and/or storing and/or transmitting the images and/or videos of at least portion of the method described herein. The images may be, e.g., photographs or illustrations of one of more steps or results of one or more steps of the method described herein. The images and/or videos may, for example, be stored on a recordable medium such as CD, flash drive or other computer-readable memory. The images and/or videos may be transmitted, for example, for display on one or more visible display units. Suitable visible display units include, for example, posters, point-of-sale displays, brochures, advertisement handouts, monitors, TV screens and/or mobile device screens. As used herein, at least a portion of the method, may refer to at least one step or a combination of steps of the method described herein, or how the substrate(s) appears before, during and/or after conducting a step of the method described herein.

In one aspect, a method is provided for displaying images and/or videos on visible display units for demonstrating, assessing and/or comparing modes of actions for skin care products or components thereof, the method comprises:
(i) selecting a first substrate and a second substrate;
(ii) combining the first substrate and second substrate in at least a first container and a second container, wherein each container has a surface layer predominately comprising the first substrate;
(iii) applying a first product to the first container and a second product to the second container, preferably wherein the first and second products to coat the surface layer in each respective container, and preferably, wherein the first and second product is different in each container;
(iv) assessing the second substrate in each of the first and second containers, wherein the second substrate is capable of changing its appearance after interacting with the products; and
(v) displaying images and/or videos of any previous step(s).

As used herein, “displaying images and/or photos of any previous step(s)”, may refer to an image and/or video of conducting at least one step or a combination of steps of the method described herein, and/or how the substrate(s) appears before, during and/or after conducting a step or more than one step of the method described herein.

In one aspect, a method is provided for recording images and/or videos for demonstrating, assessing and/or comparing modes of actions for skin care products or components thereof, the method comprises:
(i) selecting a first substrate and a second substrate;
(ii) combining the first substrate and second substrate in at least a first container and a second container, wherein each container has a surface layer predominately comprising the first substrate;
(iii) applying a first product to the first container and a second product to the second container, preferably wherein the first and second products to coat the surface layer in each respective container, and preferably, wherein the first and second product is different in each container;
(iv) assessing the second substrate in each of the first and second containers, wherein the second substrate is capable of changing its appearance after interacting with the products; and
(v) recording the images and/or video of any of the previous step(s) on a recordable medium, and optionally displaying images and/or videos of any previous step(s).

The following examples are provided to facilitate an understanding of the invention. The examples are not intended to limit the scope of the claims.

### EXAMPLES

### Example 1

This example compares the mode of action of two skin care products or components thereof.

Two equal size glass cylinder containers were used (4″L×4″W×10″H). For each container, the same first and second substrates were used. The first substate was a plurality of marbles, and the second substate were a plurality of hydro beads (see, e.g., FIG. 2). The second substrate was first added to the glass container, followed by the first substrate. The weight ratio of the amount of first substrate to the second substrate was about 1.75 to 1.

The test products used in Example 1 were:
Product A: opacified water (to represent the inventive compositions in WO 2022/136357); and
Product B: water comprising 4% of carbomer (to represent comparative products).

Product A was added to the surface layer of the first container, and Product B was added to the surface layer of the second container, and a video was taken for about 20 minutes.

The results after 20 minutes showed that Product A was able to permeate through the first substrate and then, when in contact with the second substrate, the second substrate swelled and expanded which represents the fatty acid elongation and/or lipid production deep in the stratum corneum (see, e.g., FIG. 4). By contrast, Product B only minimally permeated the surface layer, but it did not permeate through the second substrate and no swelling or expanding occurred (see, e.g., FIG. 4). Accordingly, this visually demonstrates the different modes of action for two different skin care products, and the benefits of Product A.

1. A method for demonstrating or assessing the mode of action of a skin care product or a component thereof, the method comprising:
(i) selecting a first substrate and a second substrate;
(ii) combining the first substrate and second substrate in a container, wherein the second substrate is added to the bottom of the container and then the first substate is added on top of the second substrate, and wherein a surface layer being closest to the container opening is predominately comprises the first substrate;
(iii) applying a product to the surface layer, with the product in an effective amount, preferably to coat the surface layer; and
(iv) visually assessing the second substrate,
wherein the second substrate is capable of changing its appearance after interacting with the product.

2. The method of claim 1, wherein the first substrate is a plurality of solid articles.

3. The method of claim 1, wherein the second substrate is one or more porous absorbent articles.

4. The method of claim 1, wherein the product has a viscosity of from 0 to 100 mPa·s.

5. The method of claim 1, wherein the product has a viscosity of from 101 to 40000 mPa·s.

6. The method of claim 1, wherein steps iii-iv take place from 1 minute to 90 minutes, and optionally, measuring an increase in size of the second substrate.

7. The method of claim 7, wherein the method compares modes of actions for skin care products or components thereof, the method comprises:
(i) selecting the first substrate and the second substrate;
(ii) combining each of the first substrate and second substrate in at least a first container and a second container, wherein each container has the surface layer predominately comprising the first substrate;
(iii) applying a first product to the first container and a second product to the second container, wherein the first and second products to coat the surface layer in each respective container, and wherein the first and second product is different in each container, and
(iv) assessing the second substrate in each of the first and second containers, wherein the second substrate is capable of changing its appearance after interacting with the products.

8. The method of claim 7, wherein the first substrate is a plurality of solid articles.

9. The method of claim 1, wherein the second substrate is one or more porous absorbent articles.

10. The method of claim 7, wherein the first product has a viscosity of 0 to 100 mPa·s, and the second product has a viscosity of 101 to 40000 mPa·s.

11. A method of displaying images or videos on visible display units for demonstrating, assessing or comparing modes of actions for skin care products or components thereof, the method comprises:
(i) selecting a first substrate and a second substrate;
(ii) combining each of the first substrate and the second substrate in at least a first container and a second container, wherein each container has a surface layer predominately comprising the first substrate;
(iii) applying a first product to the first container and a second product to the second container, wherein the first and second products to coat the surface layer in each respective container, and wherein the first and second product is different in each container;
(iv) assessing the second substrate in each of the first and second containers, wherein the second substrate is capable of changing its appearance after interacting with the products; and
(v) displaying images or videos of any previous steps.

12. The method of claim 11, wherein the first product has a viscosity of 0 to 100 mPa·s, and the second product has a viscosity of 101 to 40000 mPa·s.

13. A method of recording images or videos for demonstrating, assessing or comparing modes of actions for skin care products or components thereof, the method comprises:
(i) selecting a first substrate and a second substrate;
(ii) combining each of the first substrate and second substrate in at least a first container and a second container, wherein each container has a surface layer predominately comprising the first substrate;
(iii) applying a first product to the first container and a second product to the second container, wherein the first and second products coat the surface layer in each respective container, and, wherein the first and second product is different in each container,
(iv) assessing the second substrate in each of the first and second containers, wherein the second substrate is capable of changing its appearance after interacting with the products; and
(v) recording the images and/or video of any of the previous step(s) on a recordable medium, and optionally, displaying images and/or videos of any previous steps.

14. The method of claim 13, wherein the first product has a viscosity of 0 to 100 mPa·s, and the second product has a viscosity of 101 to 40000 mPa·s.

15. The method of claim 2, wherein the solid articles are at least one of marbles, pebbles, stones, or any combination thereof.

16. The method of claim 3, wherein the one or more porous absorbent articles is at least one of hydromorphic polymers, sponges, feminine hygiene products, diapers, or any combination thereof.

17. The method of claim 4, wherein the first product has the viscosity of from 1 to 50 mPa·s.

18. The method of claim 5, wherein the second product has the viscosity of from 5000 to 20000 mPa·s.

19. The method of claim 6, wherein steps iii-iv take place from 1 minute to 60 minutes, and optionally, measuring an increase in size of the second substrate.

20. The method of claim 6, wherein steps iii-iv take place from 1 minute to 30 minutes, and optionally, measuring an increase in size of the second substrate.
